(19) United States
(12) Patent Application Publication
(10) Pub. No.: US 2019/0240132 A1
(43) Pub. Date: Aug. 8, 2019

(54) Skin care combination

(71) Applicants: Kun-Lin Lee, Kaohsiung City (TW); Chung-Yu Hsieh, Kaohsiung City (TW)

(72) Inventors: Kun-Lin Lee, Kaohsiung City (TW); Chung-Yu Hsieh, Kaohsiung City (TW)

(21) Appl. No.: 16/384,986

(22) Filed: Apr. 16, 2019

(30) Foreign Application Priority Data
Jul. 26, 2018 (TW) 107125927

(51) Int. Cl. A61K 8/49; A61K 8/44; A61K 8/64; A61K 8/19; A61Q 19/00; A61K 8/27; A61K 8/67; A61K 8/36; A61K 8/55

(52) U.S. Cl. CPC A61K 8/4953; A61K 8/44; A61K 8/64; A61K 8/19; A61Q 19/00; A61K 8/553; A61K 8/67; A61K 8/673; A61K 8/675; A61K 8/361; A61K 8/27

(57) ABSTRACT

A skin care combination and manufacturing method thereof are provided. The skin care combination includes micro cluster water, ion compounds, vitamin, amino acid, carbon source and Ectoin. the micro cluster water is the water with 17 O-NMR half peak width smaller than 80 HZ. The ion compounds include one of or any combination of calcium ion, magnesium ion, potassium ion, ferrous ion, zinc ion and copper ion. The vitamin includes one of or any combination of biotin, choline chloride, vitamin B12, folic acid and the derivant thereof, inositol and the derivant thereof, D-Panthenol and the derivant thereof, vitamin B3 and the derivant thereof, vitamin B6 and the derivant thereof. The amino acid includes one of or any combination of human essential amino acid and the derivant thereof, ornithine and the derivant thereof, glutathione and the derivant thereof. The carbon source includes one of or any combination of carbohydrates and sodium pyruvate.

### BACKGROUND OF THE INVENTION

### 1. Field of the Invention

The invention relates to skin care products and more particularly to a skin care combination and a manufacturing method thereof having improved characteristics.

### 2. Description of Related Art

Conventional technology uses the active cofactors from deep ocean water which contain different microelements and can be used as collagen peptide. But in fact, the composition of deep ocean water is not stable, and there may be obvious differences in the effectiveness due to different times and sites, so there is room for improvement.

### SUMMARY OF THE INVENTION

It is therefore one object of the invention to provide a skin care combination, comprising micro cluster water, ion compounds, vitamin, amino acid, carbon source and Ectoin; wherein the micro cluster water is the water with 17 O-NMR half peak width smaller than 80 HZ; wherein the ion compounds comprises one of or any combination of calcium ion, magnesium ion, potassium ion, ferrous ion, zinc ion and copper ion; wherein the vitamin comprises one of or any combination of biotin, choline chloride, vitamin B12, folic acid and the derivant thereof, inositol and the derivant thereof, D-Panthenol and the derivant thereof, vitamin B3 and the derivant thereof, vitamin B6 and the derivant thereof; wherein the amino acid comprises one of or any combination of human essential amino acid and the derivant thereof, ornithine and the derivant thereof, glutathione and the derivant thereof; and wherein the carbon source comprises one of or any combination of carbohydrates and sodium pyruvate.

The above and other objects, features and advantages of the invention will become apparent from the following detailed description.

### DETAILED DESCRIPTION OF THE INVENTION

A skin care combination and the manufacturing method thereof of the invention are shown and illustrated respectively. The skin care combination comprises micro cluster water, ion compounds, vitamin, amino acid, carbon source, Ectoin, fatty acids, penetrant and pH regulator; wherein the skin care combination, in practice, can only comprises the micro cluster water, the ion compounds, the vitamin, the amino acid, the carbon source and the Ectoin, or one of or any combination of the fatty acids, the penetrant and the pH regulator selectively.

The pH of the skin care combination is 7.0-7.5, which is a preferable pH; in practice, the pH of the skin care combination can be 5.5 to 8.0. The micro cluster water refers to the water with 17 O-NMR half peak width smaller than 80HZ. The ion compounds comprise calcium ion, magnesium ion, potassium ion, ferrous ion, zinc ion and copper ion. The vitamin comprises biotin, choline chloride, vitamin B12, folic acid, inositol, D-Panthenol, vitamin B3 and vitamin B6. The amino acid comprises human essential amino acid, ornithine and glutathione. The carbon source comprises carbohydrates and sodium pyruvate. The fatty acids comprise omega-3 fatty acids, omega-6 fatty acids, omega-9 fatty acids and lipoic acid. The penetrant comprises propylene glycol, glycerin, butylene glycol, dipropylene glycol, sorbitol, urea, lecithin, phospholipids and synthetic ester.

The ion compounds, in practice, can selectively comprise one of or any combination of the calcium ion, the magnesium ion, the potassium ion, the ferrous ion, the zinc ion and the copper ion. The vitamin, in practice, can selectively comprises one of or any combination of the biotin, the choline chloride, the vitamin B12, the folic acid and the derivant thereof, the inositol and the derivant thereof, the D-Panthenol and the derivant thereof, the vitamin B3 and the derivant thereof, the vitamin B6 and the derivant thereof. The amino acid, in practice, can selectively comprise one of or any combination of the human essential amino acid and the derivant thereof, the ornithine and the derivant thereof, the glutathione and the derivant thereof. The carbon source, in practice, can selectively comprise one of or any combination of the carbohydrates and the sodium pyruvate. The fatty acids, in practice, can selectively comprises one of or any combination of the omega-3 fatty acids, the omega-6 fatty acids, the omega-9 fatty acids and the lipoic acid. The penetrant, in practice, can selectively comprises one of or any combination of the propylene glycol, the glycerin, the butylene glycol, the dipropylene glycol, the sorbitol, the urea, the lecithin and the derivant thereof, the phospholipids and the derivant thereof and the synthetic ester.

The calcium ion of the ion compounds is derived from calcium lactate; the magnesium ion is derived from magnesium sulfate; the potassium ion is derived from potassium chloride; the ferrous ion is derived from ferric nitrate or ferrous sulfate; the zinc ion is derived from zinc sulfate; and the copper ion is derived from copper sulfate.

The skin care combination is made from raw material components and relatively 99.56%-10% micro cluster water, 0.01%-15% ion compounds, 0.1%-15% vitamin, 0.1%-15% amino acid, 0.1%-15% carbon source, 0.01%-5% Ectoin, 0.01%-5% fatty acids, 0.1%-15% penetrant and 0.01%-5% pH regulator.

The micro cluster water, the ion compounds, the vitamin, the amino acid and the carbon source are mixed uniformly or dissolved completely at ordinary temperature to make the first mixed material; the fatty acid, the penetrant and the Ectoin are mixed uniformly or dissolved completely at ordinary temperature to make the second mixed material. The first mixed material, the second mixed material and the pH regulator are mixed uniformly and the pH is adjusted to 7.0-7.5 to obtain the skin care combination. The pH of the skin care combination can be adjusted to 5.5-8.0.

The skin care combination has high permeability and can regulate the growth of horn cells, and can increase the skin water content greatly and reduce the surface moisture evapotranspiration, and its pH is 7.0-7.5, close to human tissue fluid. Therefore, it has high biocompatibility and low exclusiveness after it permeates through the skin, so as to avoid skin allergy or irritation.

The human skin surface has a sebum membrane which can protect the skin and retain moisture, and the gaps of keratoderma are full of intercellular lipid, so the hydrophilic raw material components form the permeation barriers as limited by the molecular property of oil-water incompatibility, and relevant care components only stay on the skin surface. The penetrant of the skin care combination can overcome the osmotic resistance resulted from oil-water compatibility effectively, so as to increase the infiltration rate and penetration depth of the skin care combination in the skin, to infiltrate the care components into the skin base for moisture retention and care.

The micro cluster water of the skin care combination is also known as micromolecular cluster water, generally referring to the water cluster aggregated of less than 50 water molecules, characterized by strong permeability and long stay in cells. Therefore, the micro cluster water is favorable for entering the Aquaporin directly, so as to supply moisture rapidly to activate the cells to replenish the skin. The micro cluster water can be determined by 17 O-NMR peak full width at half maximum, the width represents the acting force among water molecules, smaller full width at half maximum represents less water molecules in the water cluster. The present methods for making micro cluster water include heating, electrolysis and magnetization.

The ion compounds of the skin care combination have different effectiveness and assistance to skin conditioning. The calcium ion is the important factor in information transfer inside and outside cells and in moisture control. If the calcium ion concentration in epidermis is too low (the skin barrier is damaged), the moisture in the skin is transferred to the horny layer, the moisture is dissipated. The ferrous ion assists in oxygen delivery. The zinc ion is related to immunoregulation. The copper ion is required for collagen formation (synthesis).

The vitamin, the fatty acid, the amino acid and the carbon source of the skin care combination can supplement the energy for cellular metabolism and provide the sources of carbon and nitrogen. Therefore, the growth rate of horn cells can be controlled effectively, the intercellular space of horny layer can supplement endogenous intercellular lipid automatically to reduce the dissipation of moisture.

The Ectoin of the skin care combination can regulate the HSP and Langerhans cell, so as to prevent the cell defects induced by external environment, and it has restorability to protect the cells against the UVA and UVB radiation injuries.

The penetrant of the skin care combination breaks through the sebum membrane of skin and the obstacle of intercellular lipid, so that the micro cluster water can get to the deep cells of skin rapidly to make up moisture and help adjust the pH of the combination to approximate human tissue fluid, so as to reduce the stimulus response during infiltration. The Ectoin resists the defects induced by UV while the micro cluster water supplements skin moisture, so as to reduce the damage to horn layer and to prolong the effective time of the skin care combination.

The skin care combination can reach deep skin in a short period of time to supplement the moisture content in skin rapidly, the water replenishing efficiency and moisture retention effect are enhanced greatly, and the permeation of other care components is accelerated, so as to enhance the effectiveness and permanence of skin care; meanwhile the growth of horn cells is conditioned, so that the skin can repair the cutin barrier spontaneously and resist the epidermis defects and discomfort induced by external environment (e.g. UV, high temperature), so as to form a complete moisture retention system for water replenishing, water retention and skin protection and care.

While the invention has been described in terms of preferred embodiments, those skilled in the art will recognize that the invention can be practiced with modifications within the spirit and scope of the appended claims.

What is claimed is:

1. A skin care combination, comprising micro cluster water, ion compounds, vitamin, amino acid, carbon source and Ectoin;
wherein the micro cluster water is the water with 17 O-NMR half peak width smaller than 80 HZ;
wherein the ion compounds comprise one of or any combination of calcium ion, magnesium ion, potassium ion, ferrous ion, zinc ion and copper ion;
wherein the vitamin comprises one of or any combination of biotin, choline chloride, vitamin B12, folic acid and the derivant thereof, inositol and the derivant thereof, D-Panthenol and the derivant thereof, vitamin B3 and the derivant thereof, vitamin B6 and the derivant thereof;
wherein the amino acid comprises one of or any combination of human essential amino acid and the derivant thereof, ornithine and the derivant thereof, glutathione and the derivant thereof; and
wherein the carbon source comprises one of or any combination of carbohydrates and sodium pyruvate.

2. The skin care combination of claim 1, wherein the skin care combination comprises one of or any combination of fatty acid, penetrant and pH regulator;
wherein the fatty acid comprises one of or any combination of omega-3 fatty acids, omega-6 fatty acids, omega-9 fatty acids and lipoic acid; and wherein the penetrant comprises one of or any combination of propylene glycol, glycerin, butylene glycol, dipropylene glycol, sorbitol, urea, lecithin and the derivant thereof, phospholipids and the derivant thereof and synthetic ester.

3. The skin care combination of claim 1, wherein the pH of the skin care combination is 5.5 to 8.0.

4. The skin care combination of claim 3, wherein the preferred pH of the skin care combination is 7.0-7.5.

5. The skin care combination of claim 4, wherein the calcium ion of the ion compounds is derived from calcium lactate; wherein the magnesium ion is derived from magnesium sulfate; wherein the potassium ion is derived from potassium chloride; wherein the ferrous ion is derived from ferric nitrate or ferrous sulfate; wherein the zinc ion is derived from zinc sulfate; and wherein the copper ion is derived from copper sulfate.

6. A skin care combination, which is made from raw material components and relatively 99.56%-10% micro cluster water, 0.01%-15% ion compounds, 0.1%-15% vitamin, 0.1%-15% amino acid, 0.1%-15% carbon source, 0.01%-5% Ectoin, 0.01%-5% fatty acids, 0.1%-15% penetrant and 0.01%-5% pH regulator;
wherein the micro cluster water, the ion compounds, the vitamin, the amino acid and the carbon source are mixed uniformly or dissolved completely at ordinary temperature to make the first mixed material;
wherein the fatty acid, the penetrant and the Ectoin are mixed uniformly or dissolved completely at ordinary temperature to make the second mixed material; and
wherein the first mixed material, the second mixed material and the pH regulator are mixed uniformly and the pH is adjusted to 5.5-8.0 to obtain the skin care combination.

7. The skin care combination of claim 6, wherein the preferred pH of the skin care combination is 7.0-7.5.
